(19) United States
(12) Patent Application Publication
(10) Pub. No.: US 2025/0171364 A1
(43) Pub. Date: May 29, 2025

(54) PRE-COLORED SLURRY FOR CERAMIC MULTI-COLOR INKJET 3D PRINTING

(71) Applicant: Ivoclar Vivadent AG, Schaan (LI)

(72) Inventors: Hendrik JOHN, Buchs (CH); Frank ROTHBRUST, Röns (AT); Sebastian KROLIKOWSKI, Richterswil (CH); Christian RITZBERGER, Grabs (CH)

(21) Appl. No.: 18/952,263

(22) Filed: Nov. 19, 2024

(30) Foreign Application Priority Data
Nov. 29, 2023 (EP) 23213107.8

(51) Int. Cl. C04B 35/488; A61C 13/00; B28B 1/00; B33Y 30/00; B33Y 40/00; B33Y 70/00; B33Y 80/00; C04B 35/626; C04B 35/638; C04B 35/64

(52) U.S. Cl. CPC C04B 35/488; A61C 13/0019; B28B 1/001; B33Y 30/00; B33Y 40/00; B33Y 70/00; B33Y 80/00; C04B 35/62655; C04B 35/638; C04B 35/64; C04B 2235/3225; C04B 2235/3246; C04B 2235/6026; C04B 2235/9661

(57) ABSTRACT

A method of producing a dental restoration by jet-printing, including the steps of jet-printing (S101) a dentin core of the dental restoration using a plurality of opaque ceramic slurries having different colors from each other; and jet-printing (S102) the tooth enamel of the dental restoration using a translucent ceramic slurry.

### CROSS-REFERENCE TO RELATED APPLICATIONS

This application claims priority to European Patent Application No. 23213107.8 filed on Nov. 29, 2023, which is incorporated herein by reference in its entirety.

### TECHNICAL FIELD

The present invention relates to a method of producing a dental restoration by jet-printing and to a production apparatus for producing a dental restoration by jet-printing.

### BACKGROUND

Nowadays, differently pre-colored, solvent-based ceramic slurries, for example made of zirconium dioxide, are selectively applied and dried drop by drop in a plurality of layers using a jet process (inkjet process). However, processing highly filled ceramic slurries places high demands on the print head used in terms of particle size, filling level, viscosity, abrasion and corrosion. Jet-printing (jetting) of aqueous ceramic slurries is an alternative to solvent-based slurries.

US 20200017699, 20120308837 and 20100249305 are directed to 3D printing and are hereby incorporated by reference in their entirety.

### SUMMARY

It is the technical task of the present invention to produce ceramic dental restorations that are true to color and have a natural appearance by means of a three-dimensional printing process.

This task is solved by subject-matter according to the independent claims. Technically advantageous embodiments are the subject-matter of the dependent claims, the description and the drawings.

According to a first aspect, the technical task is solved by a method of producing a dental restoration by jet-printing, comprising the steps of jet-printing a dentin core of the dental restoration using a plurality of opaque ceramic slurries having different colors from one another; and jet-printing the tooth enamel of the dental restoration using a translucent ceramic slurry. Aqueous or solvent-based slurries can be used as ceramic slurries. A limited number of pre-colored slurries is provided for the method in order to reduce the number of print heads required and still achieve a highly aesthetic and functional result for the dental restoration.

An opaque slurry is a slurry that does not transmit light after the sintering process and is non-transparent. In contrast, a translucent slurry is a slurry that partially transmits light after the sintering process but does not allow clear images or shapes to be recognized. The reciprocal (mutual) property to translucency is opacity (opaqueness to light). If a sintered slurry, i.e. the final ceramic, has a high translucency, it has a low opacity in the dental restoration and vice versa. Before the sintering process, the optical properties of the respective slurries are generally different. The translucency or opacity is only created by the chemical composition of the slurry in conjunction with the sintering process in the final sintered ceramic. The optical properties of translucency or opacity are therefore the result of the sintering process.

An opaque slurry comprises, for example, yttrium-stabilized ZrO₂ particles with a Y₂O₃ content of 1.5 to 4.5 mol %, preferably 2 to 4 mol % and particularly preferably 2.5 to 3.5 mol %.

A translucent slurry comprises, for example, yttrium-stabilized ZrO₂ particles with a Y₂O₃ content of 4.0 to 8.0 mol %, preferably 4.0 to 7.0 mol % and particularly preferably 4.0 to 6.0 mol %.

In a technically advantageous embodiment of the method, an yttrium content of the translucent ceramic slurry is higher than the yttrium content of one of the opaque ceramic slurries. This achieves the technical advantage, for example, that particularly suitable slurries are used to produce areas of different translucency in the sintered dental restoration, high in the tooth enamel and low in the dentin core.

In a further technically advantageous embodiment of the method, the dentin core of the dental restoration is additionally jet-printed using a colored translucent slurry. This achieves the technical advantage, for example, that the natural appearance of the dental restoration is further improved.

In a further technically advantageous embodiment of the method, the different colors of the slurries for the dentin core form a color scheme with a limited color space. The limited color space is adapted for the coloring of the dental restoration. If a color scheme were used to cover the entire color space, a higher number of print heads would be required. The production of these different slurries is also complex. This has the technical advantage, for example, that the number of print heads can be reduced and the number of slurries kept in stock can be reduced.

In a further technically advantageous embodiment of the method, an area of the dental restoration having an intermediate color value and/or intermediate translucency value is produced by mixing at least two slurries. This achieves the technical advantage, for example, that the dental restoration can be produced with color values or translucency values that do not correspond to those of the slurries used.

In another technically advantageous embodiment of the method, the mixing is implemented by generating a two-dimensional dithering pattern for a layer of the dental restoration. The dithering pattern indicates how the different slurries are arranged in a two-dimensional plane in order to generate the intermediate color value and/or intermediate translucency value. This also achieves the technical advantage, for example, that the dental restoration can be produced with a natural appearance.

In a further technically advantageous embodiment of the method, different two-dimensional dithering patterns are used in successive layers of the dental restoration. This achieves the technical advantage, for example, of preventing the formation of stripe or moiré patterns in the dental restoration.

In a further technically advantageous embodiment of the method, a print head is assigned to each receiving container for slurry. This achieves the technical advantage, for example, that the dental restoration can be printed quickly and easily.

In a further technically advantageous embodiment of the method, the dental restoration is sintered in a sintering furnace. This achieves the technical advantage, for example, that a dental restoration with high strength can be produced.

In a further technically advantageous embodiment of the method, the produced dental restoration is subjected to a drying and/or debinding step prior to a sintering process. This drying and/or debinding step can take place in a separate thermal process or is an upstream process step in the sintering process. Drying is usually carried out at temperatures of 25° C. to 200° C., preferably 30° C. to 180° C. and particularly preferably 40° C. to 150° C. In addition, the humidity can be regulated between 10 and 90%, preferably 15 to 85% and particularly preferably 20 to 80%. Debinding is usually carried out at temperatures of 50° C. to 600° C., preferably between 100° C. and 600° C. and particularly preferably at 200° C.-600° C. The heating rates are between 0.1 and 10 K/min, preferably between 0.2 and 10 K/min and particularly preferably between 0.5 and 10 K/min.

According to a second aspect, the technical task is solved by a production apparatus for producing a dental restoration by jet-printing, comprising: a plurality of receiving containers for receiving opaque ceramic slurries for a dentin core having different colors from each other; and at least one receiving container for receiving a translucent ceramic slurry for the tooth enamel. The production apparatus uses a three-dimensional multi-color printing process by means of material-jetting of ceramic slurries. The production apparatus achieves the same technical advantages as the method according to the first aspect.

In a technically advantageous embodiment of the production apparatus, the production apparatus comprises one print head per receiving container. This achieves the technical advantage, for example, that the dental restoration can be printed quickly and easily.

In a further technically advantageous embodiment of the production apparatus, the production apparatus comprises at least two receiving containers for slurry, such as five, six, seven or eight receiving containers for slurry. This achieves the technical advantage, for example, that a small number of print heads and receiving containers can be used to produce lifelike dental restorations.

In a further technically advantageous embodiment of the production apparatus, the different colors of the slurries for the dentin core form a color scheme with a limited color space. This achieves the technical advantage, for example, that a smaller number of print heads or receiving containers can be used than with a complete color scheme.

In a further technically advantageous embodiment of the production apparatus, the production apparatus comprises a dithering module for calculating intermediate color values or intermediate translucency values by mixing at least two slurries. This achieves the technical advantage, for example, that the dental restoration can be produced with color values or translucency values that do not correspond to those of the slurries used.

In a further technically advantageous embodiment of the production apparatus, the dithering module is configured to use different two-dimensional dithering patterns in successive layers of the dental restoration. This achieves the technical advantage, for example, of preventing the formation of stripe or moiré patterns in the dental restoration.

According to a third aspect, the technical task is solved by jet-printing system, comprising a production apparatus for producing a dental restoration by jet-printing; at least one of a plurality of opaque ceramic slurries for printing a dentin core having different colors from each other; and a translucent ceramic slurry for printing the tooth enamel. The jet-printing system achieves the same technical advantages as the method according to the first aspect.

In a technically advantageous embodiment of the jet-printing system at least one of the plurality of opaque ceramic slurries and/or the translucent ceramic slurry is stored in a container. This achieves the technical advantage, for example, that the opaque ceramic slurries and/or the translucent ceramic slurry can be stored reliably.

In a further technically advantageous embodiment of the jet-printing system the container is replaceable. This achieves the technical advantage, for example, that the solutions can be exchanged easily.

### BRIEF DESCRIPTION OF THE DRAWINGS

Exemplary embodiments of the invention are shown in the drawings and are described in more detail below, in which:

FIG. 1 shows an illustration of different areas of an incisor;

FIG. 2 shows a schematic illustration of a dental restoration;

FIG. 3 shows a schematic illustration of a production apparatus for producing a dental restoration by jet-printing; and

FIG. 4 shows a block diagram of a method of producing the dental restoration by jet-printing.

### DETAILED DESCRIPTION

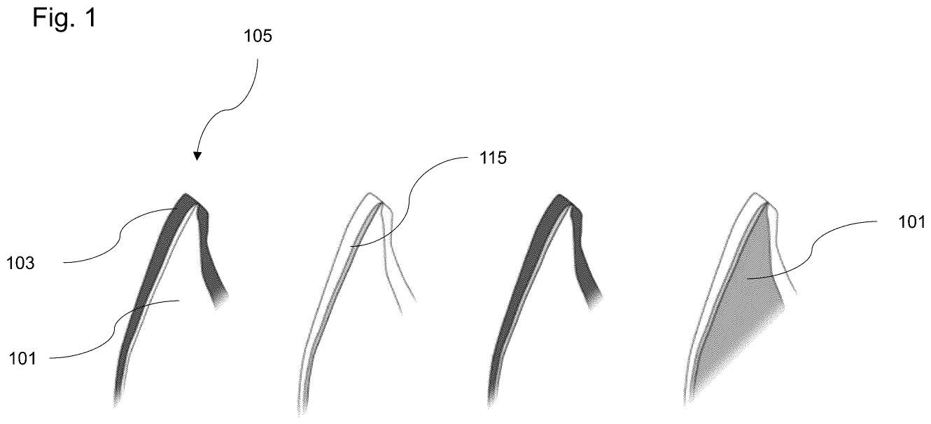

FIG. 1 shows an illustration of different areas of a tooth 105. The tooth 105 comprises an inner dentin core 101 and an outer tooth enamel 103. Between the dentin core 101 and the tooth enamel 103 there is an intermediate area 115.

The opaque, i.e., non-transparent dentin core 101 is responsible for the basic coloration of the tooth 105. This shines through the tooth enamel 103 of the incisal edge. The tooth enamel 103 is translucent. Translucency is the partial light transmittance of a body. In order to make a dental restoration look as lifelike as possible, this structure of the tooth 105 is also used in the artificial dental restoration. For this purpose, materials with different optical properties are used in the production of the dental restoration.

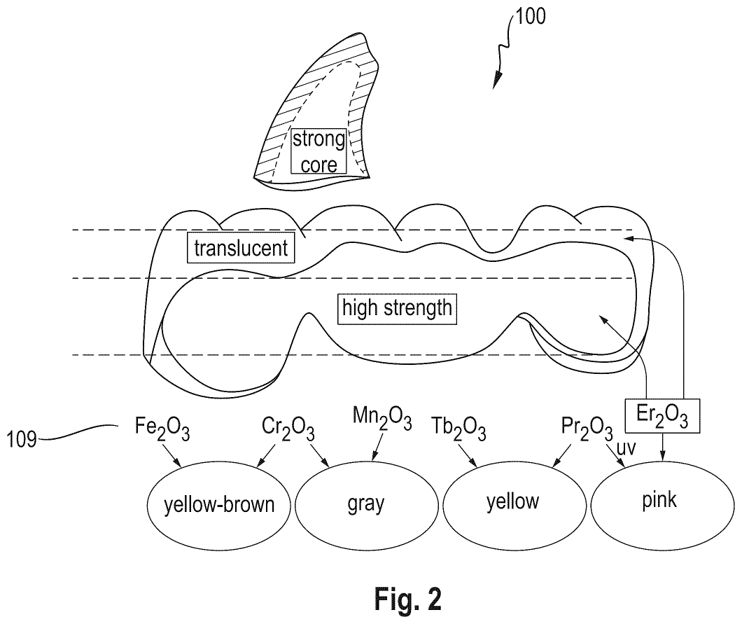

FIG. 2 shows a schematic illustration of a dental restoration 100. The dental restoration 100 serves as a dental prosthesis and is formed, for example, by a bridge, crown, veneer, inlay, onlay, abutment, full or partial prosthesis. The dental restoration 100 is built up, for example, by means of differently colored ceramic slurries, which may contain small amounts of coloring components, such as Fe₂O₃, Cr₂O₃, Mn₂O₃, Tb₂O₃, Pr₂O₃, Er₂O₃, CO₃O₄, NiO, TiO₂, CeO₂. For this purpose, these slurries are used selectively in the respective areas during the spatial production of the dental restoration 100 by means of a jet-printing process.

The dental restoration 100 is built up by successive layers that are printed on top of each other. The ceramic powders of the slurries may already be provided in predetermined colors and translucencies. Mixing these slurries results in the target tooth color and the target translucency of the dental restoration 100 in the respective spatial area.

After the selective material application of a layer of the slurry using the jet-printing process, this layer is dried without cracks by evaporating the water or solvent. What remains is a porous white body layer with a layer thickness of 1 μm to 50 μm and a density of at least 2.5 g/cm³. This process is repeated until the entire dental restoration 100 is built up spatially in layers.

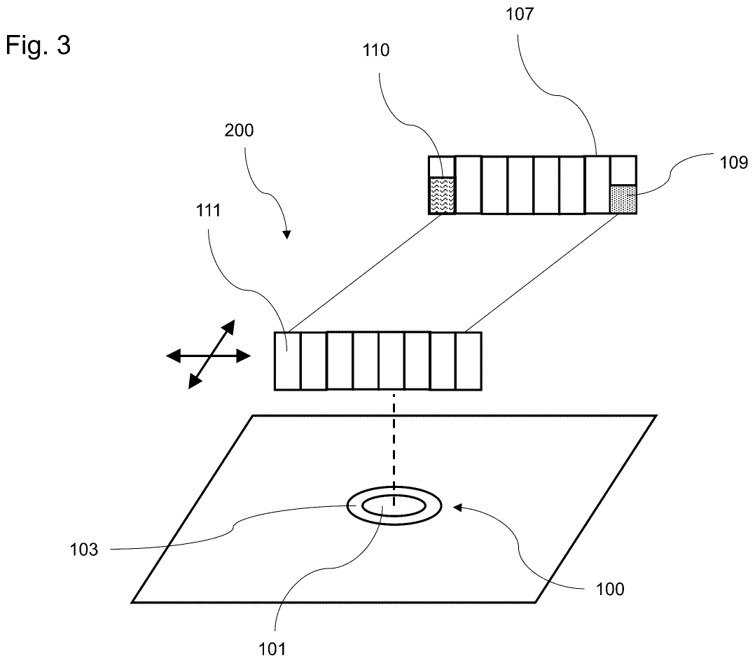

FIG. 3 shows a schematic illustration of a production apparatus 200 for producing the dental restoration 100 by jet-printing (inkjet-printing) of aqueous or solvent-based slurries 109. Pre-colored slurries 109 are processed in order to additively produce ceramic, dental multi-material and multi-color restorations 100 by means of jet-printing.

The production apparatus 200 comprises a plurality of receiving containers 107, in each of which a ceramic slurry 109 with different optical properties for the dentin core 101 is arranged. In addition, the production apparatus 200 comprises at least one receiving container 107 in which a translucent ceramic slurry 109 for producing the tooth enamel 103 is received. The production apparatus for producing the dental restoration by jet-printing; at least one of a plurality of opaque ceramic slurries for printing a dentin; and the translucent ceramic slurry for printing the tooth enamel form together a jet-printing system.

The ceramic slurries 109 are respectively applied drop by drop in several layers by means of an assigned print head 111 in order to build up the dental restoration 100 spatially in layers. The print head 111 is movable in two directions so that the slurry 109 can be printed on at any position. For selective material application, slurries 109 with a drop volume of typically 10 to 100 pL are used, the use of which eliminates the time-consuming debinding process. Electrically controlled piezo elements are used to eject the droplets. Alternatively, the bubble jet process can also be used.

The production apparatus 200 provides a reduced number of pre-colored and yttrium-doped slurries 109 to minimize the number of print heads 111 and still achieve an esthetic and functional result of the dental restoration 100.

The desired tooth color is composed and mixed from the pre-colored slurries 109. A subtractive color system is used, which spans a limited, dental color space (dental color gamut). The color mixture, the three-dimensional halftoning or dithering of these pre-colored slurries 109 in various ratios is then created in the special dental color gamut, which covers the common tooth colors, but not all colors.

Differently colored slurries 109 are selectively applied in one layer using a 3D dithering algorithm, which is executed by a dithering module 113. For this purpose, the dithering module 113 comprises a processor for executing the 3D dithering algorithm and a digital data memory for storing the calculated mixing ratios. The processor comprises any hardware system, component, or mechanism that processes data, signals, or other information. A processor may comprise a system with a central processing unit (CPU), multiple processing units (MPU), dedicated electrical circuitry to achieve functionality, or other systems. The data memory may comprise hard disks, flash memory cards, random access memories (RAN), read-only memories (ROM).

During dithering, the slurries 109 of different colors are selectively applied in a certain ratio and two-dimensional print pattern in the print plane using the jet-printing process. The 3D dithering algorithm also calculates that non-identical two-dimensional dithering patterns are applied in several layers on top of each other. This prevents optical artifacts such as stripes or moiré patterns on vertical surfaces. The color mixing, three-dimensional halftoning or dithering of these pre-colored slurries 109 in different ratios is created within the special dental color space (gamut), which covers the common tooth colors, but not all general colors.

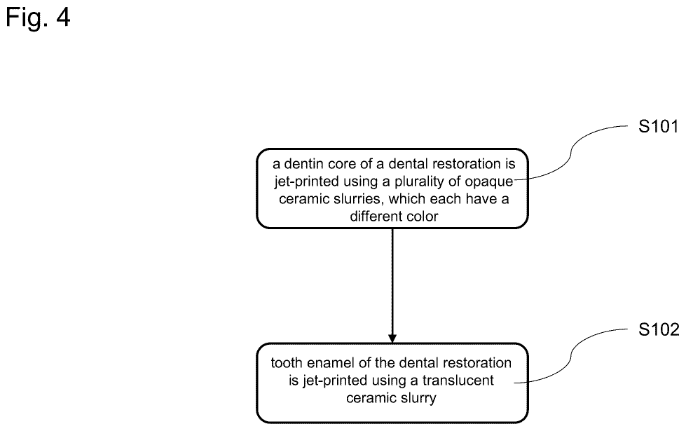

FIG. 4 shows a block diagram of a method of producing the dental restoration 100 by jet-printing. In step S101, the dentin core 101 of the dental restoration 100 is jet-printed using a plurality of opaque ceramic slurries 109, which each have a different colors. In step S102, the tooth enamel 103 of the dental restoration 100 is jet-printed by means of a translucent ceramic slurry 109. The dental restoration 100 thus built up is then sintered in a sintering furnace.

There are slurries 109, which are used to build up the tooth enamel 103 of the incisal edge, and slurries 109, which are used to build up the dentin core 101. The difference between these slurries 109 lies in the translucency of the material, which is many times higher for the tooth enamel 103 than for the dentin core 101.

For example, slurries 109 can be used in seven different basic colorations, two of which are intended for the tooth enamel 103, and an additional support material 110 for support structures. The support material is made of organic components, such as waxes, paraffins or carbon black slurries.

| | |
|---|---|
| 1 | White, highly translucent |
| | (incisal edge/area), higher Y-doping content |
| 2 | Yellow-brown, highly translucent |
| | (incisal edge/area), higher Y-doping content |
| 3 | White, opaque, high-strength |
| | (dentin area) |
| 4 | Pink, translucent, strong |
| | (dentin area, also suitable for detailing the incisal |
| | edge) |
| 5 | Gray, translucent, strong |
| | (dentin area, also suitable for detailing the incisal |
| | edge) |
| 6 | Yellow, translucent, strong |
| | (dentin area) |
| 7 | Yellow-brown, opaque, high-strength |
| | (dentin area) |
| 8 | Support material |

In another embodiment, there is only a single translucent slurry 109 and five different slurries 109 that are provided for coloring the dentin core 101. This has the technical advantage that seven print heads 111 (including support material 110) are already sufficient.

| | |
|---|---|
| 1 | Incisal edge, highly translucent (8-9 Y) |
| | (incisal edge/area) |
| 2 | White, opaque, high-strength |
| | (dentin area) |
| 3 | Pink, translucent, strong |
| | (dentin area, also suitable for detailing the incisal |
| | edge) |
| 4 | Gray, translucent, strong |
| | (dentin area, also suitable for detailing the incisal |
| | edge) |
| 5 | Yellow, translucent, strong |
| | (dentin area, also suitable for detailing the incisal |
| | edge) |
| 6 | Yellow-brown, opaque, high-strength |
| | (dentin area) |
| 7 | Support material |

The dentin core 101 is responsible for the basic coloration and shines through the tooth enamel 103 of the incisal edge. In extreme cases, the incisal edge is colorless and highly translucent. For this purpose, specially pre-colored and yttrium-doped slurries 109 are provided for coloring and for the tooth-specific core/shell build-up (dentin/incisal edge).

In the case of ZrO₂ slurries 109, different yttrium-doped ceramic powders (3 mol % yttrium-3Y-TZP, 4 mol % yttrium-4Y-TZP, 5 mol % yttrium-5Y-TZP) can be used for the different strengths. The yttrium doping is also responsible for the degree of translucency. The different yttrium-doped ceramic powders have different properties.

$$\begin{matrix}3 Y - TZP = low translucency / high strength \\ 4 Y - TZP = medium translucency / medium strength \\ 5 Y - TZP = high translucency / low strength\end{matrix}$$

However, if different pre-colored slurries 109 were used, all of which also having different translucency and/or mechanical properties, a separate print head 111 would be used for each of these slurries 109. If, for example, a general four-color scheme is used to cover the entire color space and slurries with different translucencies or strengths are also used in three variants, 3×4=12 slurries 109 in 12 print heads are required. The production of these different slurries 109 is also complex.

The pre-colored slurries 109 with their different yttrium contents should be adjusted for uniform sintering behavior. The sintering behavior of the individual layers can be adjusted by the targeted addition of sintering activators or sintering inhibitors. Sintering activators are, for example, Zn²⁺ or Mg²⁺ ions that can be added to the coloring solution in the form of soluble salts, such as Zn(NO₃)₂*6H₂O or Mg(NO₃)*H₂O. Sintering inhibitors are e.g. Al³⁺ or Y³⁺, which can be added to the coloring solution in the form of soluble salts such as Al(NO₃)₃*9H₂O or Y(NO₃)₃*6H₂O.

All the features explained and shown in connection with individual embodiments of the invention can be provided in different combinations in the subject-matter according to the invention in order to simultaneously realize their advantageous effects.

All method steps can be implemented by devices that are suitable for executing the respective method step. All functions performed by the features of the subject-matter can be a method step of a method.

The scope of protection of the present invention is given by the claims and is not limited by the features explained in the description or shown in the figures.

### REFERENCE LIST

100 Dental restoration
101 Dentin core
103 Tooth enamel
105 Tooth
107 Receiving container
109 Slurry
110 Support material
111 Print head
113 Dithering module
115 Intermediate area

1. A method of producing a dental restoration by jet-printing, comprising
jet-printing a dentin core of the dental restoration using a plurality of opaque ceramic slurries having different colors from each other; and
jet-printing an enamel of the dental restoration using a translucent ceramic slurry.

2. The method according to claim 1,
wherein an yttrium content of the translucent ceramic slurry is higher than an yttrium content of one of the plurality of opaque ceramic slurries.

3. The method according to claim 1,
wherein the dentin core of the dental restoration is additionally jet-printed using a colored translucent slurry.

4. The method according to claim 1,
wherein the different colors of the slurries for the dentin core form a color scheme with a limited color space.

5. The method according to claim 1,
wherein an area of the dental restoration having an intermediate color value and/or intermediate translucency value is produced by mixing at least two slurries.

6. The method according to claim 5,
wherein the mixing is implemented by generating a two-dimensional dithering pattern for a layer of the dental restoration.

7. The method according to claim 6,
wherein different two-dimensional dithering patterns are used in successive layers of the dental restoration.

8. The method according to claim 1,
wherein a print head is assigned to each receiving container for the slurries.

9. The method according to claim 1,
wherein the produced dental restoration is subjected to a drying and/or debinding step prior to a sintering process.

10. The method according to claim 1,
wherein the dental restoration is sintered in a sintering furnace.

11. A production apparatus for producing a dental restoration by-jet printing, comprising:
a plurality of receiving containers for receiving opaque ceramic slurries for a dentin core having different colors from each other; and
at least one receiving container for receiving a translucent ceramic slurry for the tooth enamel.

12. The production apparatus according to claim 11,
wherein the production apparatus comprises one print head per each of the plurality of receiving containers.

13. The production apparatus according to claim 11,
wherein the different colors of the slurries for the dentin core form a color scheme with a limited color space.

14. The production apparatus according to claim 11,
wherein the production apparatus comprises a dithering module for calculating intermediate color values and/or intermediate translucency values by mixing at least two slurries.

15. The production apparatus according to claim 14,
wherein the dithering module is configured to use different two-dimensional dithering patterns in successive layers of the dental restoration.

16. A jet-printing system, comprising:
a production apparatus for producing a dental restoration by jet-printing;
at least one of a plurality of opaque ceramic slurries for printing a dentin core having different colors from each other; and
a translucent ceramic slurry for printing the tooth enamel.

17. The jet-printing system according to claim 16,
wherein at least one of the plurality of opaque ceramic slurries and/or the translucent ceramic slurry is stored in a container.

18. The jet-printing system according to claim 17,
wherein the container is replaceable.
